(19) United States
(12) Patent Application Publication
(10) Pub. No.: US 2011/0034782 A1
(43) Pub. Date: Feb. 10, 2011

(54) APPARATUS FOR BODY SURFACE STIMULATION TREATMENT, PROGRAM FOR BODY SURFACE STIMULATION TREATMENT, AND COMPUTER-READABLE RECORDING MEDIUM HAVING RECORDED THE SAME

(75) Inventors: Masaru Sugimachi, Osaka (JP); Atsunori Kamiya, Osaka (JP); Kenji Sunagawa, Fukuoka (JP)

Correspondence Address: GRIFFIN & SZIPL, PC, SUITE PH-1, 2300 NINTH STREET, SOUTH, ARLINGTON, VA, 22204, US

(73) Assignee: FUJIKIN CORPORATION, Osaka (JP)

(21) Appl. No.: 12/095,629

(22) PCT Filed: Nov. 29, 2006
(86) PCT No.: PCT/JP2006/323819

§ 371 Date: Oct. 25, 2010

(30) Foreign Application Priority Data
Nov. 30, 2005 (JP) 2005-345307

(51) Int. Cl. A61B 5/00; A61N 1/00; A61F 7/00; A61H 1/00

(52) U.S. Cl. 600/301; 607/115; 607/96; 601/1

(57) ABSTRACT

[Problems] To provide an apparatus for a body surface stimulation treatment capable of overcoming the uncertainty in the existing body surface stimulation therapy and exhibiting stable therapeutic effects.

[Means for Solving Problems] An apparatus for a body surface stimulation treatment (10) which comprises a body surface stimulation unit (16) whereby the body surface of a patient is physically or chemically stimulated, a vital reaction quantitative evaluation unit (12) whereby the vital reaction of the patient is quantitatively evaluated, and a body surface stimulation regulation unit (14) whereby the stimulation properties imparted to the body surface tissues of the patient by the body surface stimulation unit (16) are regulated depending on the vital data obtained by the vital reaction quantitative evaluation unit (12).

### TECHNICAL FIELD

This invention relates to an apparatus for body surface stimulation treatment that implements relevant therapy by using a vital reaction exerted on a patient's body surface stimulation.

### BACKGROUND ART

The living body (man) possesses reflection that reacts to various stimulations from his body surface. In the body surface tissues such as, for example, the skin, the mucous membrane, the muscle, the sinew, the periosteum, the joint capsule, and the ligament, sensors that sense various simulations (somatic sensation receptors) exist. The various sensors sense the physical states (mechanical state, temperature, etc.) and the chemical states (concentrations of physiological substances, etc.) of the body surface tissues and transmit the acquired information to the brain (central nervous system) via the afferent nerve (spinal tracts, etc.). The brain that has received this information regulates the organic functions (the heart, the blood vessels, the lungs, the gastrointestine, the vesia urinaria, the sweat gland, the nerve, etc.) by dint of the efferent nerve (chiefly the autonomic nerve). As a result, the viscera are caused to react reflectively on the various stimulations from the body surface. This reaction is called somatic visceral reflex, for example.

Part of the organic reactions that are exerted on such body surface stimulations has already been adopted for use in therapy. In the case of abdominalgia that is caused by convulsive constriction of the gastrointestine, for example, a warm temperature stimulation applied to the abdominal skin represses gastrointestinal motion reflectively and alleviate the pain. When a mechanical stimulation is applied to the perineal skin of a patient of chronic myelonic injury making urination difficult, it enables the patient to urinate (refer to Non-Patent Document 1). Further, when the acupuncture (electric acupuncture, laser acupuncture, etc), moxa cautery, digital compression, etc. are considered medically and physiologically, it is thought that they are what utilize the visceral reaction exerted on the body surface stimulations.

[Non-Patent Document 1] “Standard Physiology” written by Toshinori Hongo, published by K. K. Igaku Shoin, on Oct. 1, 1996, p. 212-223, p. 366-685.

### DISCLOSURE OF THE INVENTION

### Problem to be Solved by the Invention

The therapy that utilizes the vital reaction exerted on such body surface stimulations, however, has a problem of entailing a difficulty in manifesting an authentic therapeutic effect because the treatments are carried out without objectively and quantitatively evaluating the therapeutic effects. Though the electric acupuncture, for example, possibly manifests a therapeutic effect at times, it reveals low therapeutic certainty and incurs difficulty in manifesting the optimum effect because the sites of stimulations and the characteristics of stimulations (amount of electric current, voltage, duration, etc) are decided by the subjective opinion of a relevant artisan.

This invention has been produced for the purpose of solving such problems as described above and is aimed at overcoming the uncertainty of the conventional body surface stimulation treatments and providing an apparatus for body surface stimulation treatments that can manifest a stable therapeutic effect.

### Means for Solving Problem

This invention has solved the aforementioned problems with an apparatus for body surface stimulation treatments that is characterized by comprising a body surface stimulation unit for physically or chemically stimulating a patient's body surface tissue, a vital reaction quantitative evaluation unit for quantitatively evaluating the patient's vital reaction, and body surface stimulation regulation unit for regulating the stimulation properties exerted on the patient's body surface tissue with the aforementioned body surface stimulation unit based on the vital data acquired with the aforementioned vital reaction quantitative evaluation unit.

The “body surface tissue” to which this invention pertains exists in the skin, the mucous membrane, the subcutaneous tissues, etc. and gets excited by mechanical deformation, temperature change, etc. exerted mainly from outside (superficial receptor and cutaneous receptor) and exists in the deep regions of the muscle, the sinew, the joint capsule, etc and gets excited by stimulations generally caused by the patient's own motion (deep receptor and endermic receptor).

According to this invention, it is made possible to effect the regulation of a body surface stimulation in the form of a closed loop and continuously with the vital reaction quantitative evaluation unit, the body surface stimulation regulation unit, and the body surface stimulation unit. This invention, therefore, is enabled to overcome the uncertainty of the conventional body surface stimulation treatment and manifest a stable therapeutic effect.

This invention also relates to an apparatus for body surface stimulation treatment that is characterized by the aforementioned vital reaction consisting in the patient's autonomic nerve activity, the change of the visceral function caused by the autonomic nerve activity, or the vital signal reflecting the change of the visceral function or the patient's respiration.

The autonomic nerve activity (sympathetic nerve activity or vagus nerve activity) is very important for the therapy of pathologic exacerbation, though it does not necessarily evolve to a subjective symptom unlike such a thing as pain. Since the autonomic nerve reacts for several seconds to several minutes to each of the body surface stimulations, however, it is unusually complicated and moreover difficult for a man to implement manually the regulation of a body surface stimulation. In this respect, the apparatus for body surface stimulation treatment of this invention is enabled to control the autonomic nerve not manually but automatically by having the body surface stimulation regulated in the form of a closed loop and continuously. Incidentally, while the vital reaction that is measured and quantitatively evaluated by the vital reaction quantitative evaluation unit may be an autonomic nerve activity itself, the autonomic nerve activity can be indirectly evaluated in the light of the change of the visceral function (the heart, for example) caused by the autonomic nerve activity and the vital signal (heart rate, for example) reflecting the change of this visceral function.

The apnea of an infant is a dangerous morbidity that is made by hypoxia to turn up in death. In a sudden infant death syndrome by which 400-500 person/year of infants dies, though all these sudden deaths are not definitely ascribable to the apnea of the infants, the arousal reaction, which is a protective mechanism that enables recovery from apnea caused during the course of sleep, is thought to derive retardation from a certain reason (premature, infection, stricture of the respiratory tract, etc.), further the state of hypoxia, result in repressing respiration, fall in a vicious circle, and invite death. While it is empirically known that an infant suffering from apnea is enabled to restore respiration by the stimulation of the skin (particularly the stimulation of the sole), it is difficult for a man to control his respiration throughout the whole course of sleep at night. In this point, the apparatus for body surface stimulation treatment contemplated by this invention enables the respiration to be controlled not manually but automatically by having the regulation of the body surface stimulation implemented in the form of a closed loop and continuously.

This invention relates to a apparatus for body surface stimulation treatment characterized by the aforementioned vital reaction quantitative evaluation unit being a neuroelectric meter capable of measuring the aforementioned autonomic nerve activity, an electrocardiographic meter capable of measuring at least heart rate and electrocardiographic wave form as the aforementioned vital signal, a blood flow meter capable of measuring at least pulse wave form as the aforementioned vital signal, or a blood pressure meter capable of measuring at least blood pressure as the aforementioned vital signal or by being a respiration monitor device capable of measuring the aforementioned respiratory kinetics (minute ventilation volume, respiration rate, tidal air, concentrations of oxygen and carbon dioxide pertaining to respiration, pulmonary compliance), a respiratory flow meter, impedance prestimography, an endermic oxygen gas monitor, or a respiratory gas concentration measurement device.

Generally, it is known that the increase of heart rate reflects an increase of sympathetic nerve activity and a decrease of vagus nerve activity and the decrease of heart rate reflects a decrease of sympathetic nerve activity and an increase of vagus nerve activity. Also, the indexes of sympathetic nerve activity and vagus nerve activity (the frequency component approximating an RR interval of 0.25 Hz) can be found by the frequency analysis of the wave form of an electrocardiogram (Fourier transform, autoregression method, complex demodulation method, etc.). When an electrocardiographic meter is adopted as the vital reaction quantitative evaluation unit that is contemplated by this invention, therefore, the autonomic nerve activity can be evaluated by measuring the heart rate and the wave form of an electrocardiogram.

Generally, it is also known that the increase of pulse rate found from the pulse wave reflects an increase of sympathetic nerve activity and a decrease of vagus nerve activity and the decrease of pulse rate reflects a decrease of sympathetic nerve activity and an increase of vagus nerve activity. When a local continuous blood flow meter is adopted as the vital reaction quantitative evaluation unit contemplated by this invention, therefore, the autonomic nerve activity can be evaluated by measuring the form of a pulse wave.

Further, as the vital reaction quantitative evaluation unit contemplated by this invention, a neuroelectric meter that is capable of measuring the aforementioned autonomic nerve activity can be adopted. The apparatus for body surface stimulation treatment that is so configured as described above enables the autonomic nerve activity itself to be measured by a minute electrocardiographic method.

Further, as described above, the increase of pulse rate reflects an increase of sympathetic nerve activity and a decrease of vagus nerve activity and the decrease of pulse rate reflects a decrease of sympathetic nerve activity and an increase of vagus nerve activity. Then, the indexes of sympathetic nerve activity and vagus nerve activity generally can be found by the frequency analysis of the wave form of blood pressure (Fourier transform method, autoregression method, complex demodulation method, etc.). When a blood pressure meter is adopted as the vital reaction quantitative evaluation unit contemplated by this invention, the autonomic nerve activity can be evaluated by measuring pulse rate and blood pressure (wave form).

This invention relates to an apparatus for body surface stimulation treatment that is characterized by the aforementioned body surface stimulation unit being an electric stimulation means adapted to stimulate electrically the body surface tissue of a pertinent patient. By the configuration consequently produced, it is made feasible to improve the abnormalities of autonomic nerve such as chronic cardiac failure and ischemic heart disease, namely the abnormal increase of sympathetic nerve activity and the abnormal decrease of vagus nerve activity, by an electric stimulation.

The interiors of cells that constitute nerve and sinew in a static state have a potential difference of −70-−80 mV relative to the exteriors thereof. When this potential difference decreases by several mV to several tens of mV and consequently exceeds critical depolarization, the cells are excited and made to generate action potential. The flow of electric current to a living body (the act of exerting an electric stimulation) is one of the methods for exciting nerve cells.

This invention further relates to a apparatus for body surface stimulation treatment that is characterized by the aforementioned electric stimulation means being adapted to enable regulation of at least one factor selected from among voltage, electric current, number of cycles, frequency of stimulation, and duration of stimulation. By the configuration thus produced, it is made possible to vary influence on a living body and enable therapy of high accuracy by regulating the electric stimulation property in conformity with the vital reaction.

This invention also pertains to a apparatus for body surface stimulation treatment that is characterized by the aforementioned body surface stimulation unit being a thermal stimulation unit adapted to stimulate thermally the body surface tissue of a relevant patient.

Generally, the ambient temperature is known to vary an autonomic nerve activity and the sympathetic nerve activity is inevitably strained when the room temperature is unduly high or unduly low. Particularly during the course of a patient's sleep, the regulation of ambient temperature gains in effectiveness and the patient possibly fails to vary air-conditional setting because the patient's emotion is degraded as compared with the daytime and his autonomic nerve activity becomes sensitive to the ambient temperature. By providing a thermal stimulation unit that is adapted to stimulate the body surface tissue of a living body by thermal shock or change of air pressure, it is made possible to improve the abnormalities of autonomic nerve such as chronic cardiac failure and ischemic heart disease, namely an abnormal increase of sympathetic nerve activity and an abnormal decrease of vagus nerve activity, by such a physicochemical stimulation as thermal shock or change of air pressure.

This invention relates also to a apparatus for body surface stimulation treatment that is characterized by the aforementioned body surface stimulation unit being a mechanical stimulation means adapted to stimulate mechanically the body surface tissue of a relevant patient. With the configuration thus produced, it is made possible to improve the abnormalities of autonomic nerve such as chronic cardiac failure and ischemic heart disease, namely an abnormal increase of sympathetic nerve activity and an abnormal decrease of vagus nerve activity, by a mechanical stimulation.

This invention also relates to a apparatus for body surface stimulation treatment that is characterized by the aforementioned mechanical treatment means being adapted to enable regulation of at least one factor selected from among position of stimulation, intensity of stimulation, frequency of stimulation, and duration of stimulation. According to the configuration thus produced, it is made possible to vary the influence on a living body and enable therapy of high accuracy by regulating the mechanical stimulation property in conformity with the vital reaction.

This invention further relates to a apparatus for body surface stimulation treatment that is characterized by the aforementioned body surface stimulation regulating means being adapted to enable communication with the aforementioned vital reaction quantitative evaluation unit or the aforementioned body surface stimulation unit via radio communication.

According to the configuration thus produced, it is made possible to enable prevention-therapy of such diseases of the circulatory system as arrhythmia and abnormality of circulatory kinetic and permit therapy in the case of emergency by transmitting vital data by radio communication from the vital reaction quantitative evaluation unit (such as, for example, an electrocardiographic meter, a local continuous blood flow meter, a blood pressure meter, or a neuroelectric meter) to the body surface stimulation treating means (such as, for example, a personal computer) installed in a medical organ such as a hospital located at a remove place and remote controlling the body surface stimulation regulation unit (such as, for example, an electric acupuncture device) by radio communication emitted from the source. It is consequently made possible, for example, to enable therapy and prevention of lethal arrhythmia during the onset of myocardial infarct taking place at a place like a patient's own home other than a hospital that is difficult of treatment by the existing therapy.

This invention relates also to a apparatus for body surface stimulation treatment that is characterized by the aforementioned body surface stimulation regulation unit possessing a function of outputting a stimulation condition datum and at least one stimulation condition candidate value formed by performing a specific calculation on the aforementioned stimulation condition datum, a function of retrieving the optimum vital data most suitable for the purpose of therapy from among the vital data acquired from the aforementioned vital reaction quantitative evaluation unit in conformity with the aforementioned stimulation condition datum or the output of the aforementioned stimulation condition candidate value, and a function of setting as a new stimulation condition datum the aforementioned stimulation condition datum or the aforementioned stimulation condition candidate value pertaining to the aforementioned optimum vital data.

It is difficult to have an industrial control algorithm unfit for vital signal control diverted per se for use in a body surface stimulation treatment device because the vital response to a body surface stimulation is occasionally very complicated and exhibits intense time variation. By the preceding configuration, it is made possible to enable the vital data obtained by a vital reaction quantitative evaluation unit to approximate rapidly and competently the optimum vital data most suitable for the purpose of therapy.

This invention also relates to a apparatus for body surface stimulation treatment characterized by the aforementioned body surface stimulation regulation unit possessing a function of continuing the stimulation to the body surface tissue of a patient when the vital data obtained by the aforementioned vital reaction quantitative evaluation unit fits the purpose of therapy and discontinuing the stimulation to the body surface tissue of the patient when the vital data obtained by the aforementioned vital reaction quantitative evaluation unit does not fit the purpose of therapy.

By the configuration of this nature, it is made possible to simplify and facilitate control and enable the vital data obtained by the vital reaction quantitative evaluation unit to approximate the state most suitable for the purpose of therapy.

This invention relates also to a program for body surface stimulation treatment characterized by causing a body surface stimulation treatment device to implement (1) a process for physically or chemically stimulating the body surface tissue of a patient with a body surface stimulation unit, (2) a process of quantitatively evaluating the vital reaction of the patient with a vital reaction quantitative evaluation unit, and (3) a process of regulating the stimulation property exerted to the body surface tissue of the patient with the aforementioned body surface stimulation unit based on the vital data obtained by the process (2).

This invention further relates to a program for body surface stimulation treatment characterized by the aforementioned process (3) comprising a step of outputting a stimulation condition datum and at least one stimulation condition candidate datum formed by performing a specific calculation on the aforementioned stimulation condition datum, a step of retrieving the optimum vital data most suitable for the purpose of therapy from the vital data acquired by the aforementioned vital reaction quantitative evaluation unit in conformity with the aforementioned stimulation condition datum or the output of the aforementioned stimulation condition candidate datum, and a step of setting the aforementioned stimulation condition datum or the aforementioned stimulation condition candidate datum concerning the aforementioned optimum vital data as a new stimulation condition datum.

This invention also relates to a program for body surface stimulation treatment characterized by the aforementioned process (3) comprising a step of continuing the stimulation to the body surface tissue of a patent by the aforementioned body surface stimulation unit when the vital data obtained by the aforementioned vital reaction quantitative evaluation unit fits the purpose of therapy and discontinuing the stimulation to the body surface tissue of the patient by the aforementioned body surface stimulation unit when the vital data obtained by the aforementioned vital reaction quantitative evaluation unit does not fit the purpose of therapy.

This invention also relates to a computer-readable recording medium that has recorded the aforementioned program for body surface stimulation treatment.

### EFFECT OF THE INVENTION

The apparatus for body surface stimulation treatment according to this invention possesses an outstanding effect of overcoming the uncertainty of the conventional body surface stimulation treatment and manifesting a stable therapeutic effect.

### MODE(S) FOR CARRYING OUT THE INVENTION

### Modes for Carrying Out the Invention

First, the apparatus for body surface stimulation treatment involved in the mode of embodiment of this invention will be explained in outline below.

FIG. 1 is a block diagram illustrating the main configuration of a apparatus for body surface stimulation treatment 10 that is involved in the mode of embodiment of this invention.

The apparatus for body surface stimulation treatment 10 involved in the present mode of embodiment is configured by being furnished with a vital reaction quantitative evaluation unit 12, a body surface stimulation regulation unit 14, and a body surface stimulation unit 16.

The vital reaction quantitative evaluation unit 12 is so constructed as to enable measurement and quantitative evaluation of the vital reactions of a patient, namely autonomic nerve activity (sympathetic nerve activity and vagus nerve activity), changes of visceral functions (such as the heart, the blood vessels, the lungs, the gastrointestine, the vesica urinaria, the perspiratory glands, etc.), vital signals (such as heart rate, pulse, blood pressure, etc.) reflecting the changes of the visceral functions, and respiration and permit output of the vital reactions resulting from the measurement and quantitative evaluation as vital data.

The body surface stimulation regulation unit 14 is so constructed as to compare the vital data obtained by the vital reaction quantitative evaluation unit 12 with the vital data aimed at by therapy, regulate stimulation properties imparted to the body surface tissue of the patient in accordance with the result of the comparison, and output the stimulation properties subsequent to the regulation as an instruction data.

The body surface stimulation unit 16 is adapted to implement therapy on the patient by imparting a physical or chemical stimulation to the body surface tissue of the patient based on the instruction data obtained from the body surface stimulation adjustment means 14.

The apparatus for body surface stimulation treatment 10 relies on the vital reaction quantitative evaluation unit 12, the body surface stimulation regulation unit 14, and the body surface stimulation unit 16 to implement the regulation of the body surface stimulation in the form of a closed loop and continuously.

Now, the apparatus for body surface stimulation treatment involved in Examples 1-4 of this invention will be specifically explained below with reference to the accompanying drawings.

### Example 1

FIG. 2 is a drawing illustrating in type section a apparatus for body surface stimulation treatment 20 according to Example 1 of this invention and FIG. 3 is a block diagram illustrating the main configuration of the apparatus for body surface stimulation treatment 20.

This apparatus for body surface stimulation treatment 20 is configured by being furnished with an electrocardiographic meter 22 corresponding to the aforementioned vital reaction quantitative evaluation unit 12, a regulating device 24 corresponding to the aforementioned body surface stimulation regulation unit 16, and an electric acupuncture means 16 corresponding to the aforementioned body surface stimulation unit 16.

The electrocardiographic meter 22 is provided with a plurality of electrodes 22A capable of being mounted on the body surface of a patient (man) 28. The electrocardiographic meter 22 measures such vital signals as heart rate and electrocardiographic wave form of the patient 28 through the medium of the plurality of electrodes 22A and transmits them as vital data to the regulation device 24.

The regulation device 24 compares such vital data as heart rate and electrocardiographic wave form received from the electrocardiographic meter 22 with vital data (such as, for example, adequate heart rate and electrocardiographic wave form) aimed at by therapy, regulates the properties (such as, for example, voltage, electric current, number of cycles, frequency of stimulation, duration of stimulation, etc.) of a stimulation (electric simulation in this case) imparted to the tissue of the body surface (leg in this case) of the patient 28 in accordance with the result of the comparison, and transmits the properties of stimulation subsequent to the regulation as an instruction data.

The electric acupuncture device 26 is provided with an acupuncture electrode 26A and is adapted to implement therapy on the patent 28 by electrically stimulating his body surface tissue based on the instruction data received from the regulation device 24.

The interior of the tissue that constitutes the nerves and the muscles, while standing in a resting state, exhibits a potential difference of −70-−80 mV relative to the exterior thereof. When this potential difference decreases by several mV to several tens of mV and exceeds the critical depolarization, the tissue is excited and made to generate an action potential. The fact of causing flow of electric current (the fact of imparting an electric stimulation) to a living body is one of the methods for exciting nerve cells and is widely known to possess a therapeutic effect.

The apparatus for body surface stimulation treatment 20 involved in Example 1, by causing the electrocardiographic meter 22 to measure, determine and evaluate again the changes of the pulse rate and the electrocardiographic wave form of the patient 2 induced by an electric stimulation, is enabled to form a closed loop of electrographic meter 22→regulation device 24→electric acupuncture device 26 and effect automatic adequation of the pulse rate and the electrocardiographic of the patient 28.

FIG. 4 shows the results of an experiment that was performed by the present inventor by means of the apparatus for body surface stimulation treatment 20. Incidentally, FIG. 4 is a graph illustrating the relation between the pulse rate measured with the electrocardiographic meter 22 of the apparatus for body surface stimulation treatment 20 and the voltage (stimulation voltage) and the frequency (stimulation frequency) of the electric signal issued from the electric acupuncture device 26.

In this experiment, i.e., an animal (rabbit) test under the control of venous anesthesia and artificial respiration, the body blood pressure was continuously measured with a pressure catheter inserted through the right femoral artery and the electrocardiogram was measured as well with the electrocardiographic meter 22 and the pulse rate was monitored and evaluated in real time based on the consequently obtained results.

The outer side of the front face about 5 mm below the left stile bone (corresponding to the pit called “Ashi Sanri” in the Human Oriental Medicine) and the outer side of the front face about 1 cm above the articulation of foot were electrically stimulated by the electric acupuncture device 26 through the medium of an acupuncture electrodes 26A (made of stainless steel) inserted to a depth of about 8 mm in the relevant outer sides.

The electric acupuncture device 26, by being externally controlled with the DA signal from a personal computer serving as the adjusting device 24, was enabled to effect automatic regulation of the stimulation voltage and the stimulation frequency in conformity with the actual evaluation value of pulse rate so as to lower the pulse rate. Incidentally, the regulation of the stimulation voltage and the stimulation frequency was carried out by means of the Algorithm 1 (searching control) that will be explained herein below.

FIG. 5 (A) is a drawing illustrating in type section the flow of the process of Algorithm 1 (searching control).

At the step S1, the body surface insulation regulation means 14 (the regulation device 24 in Example 1) outputs an initial value of stimulation condition as an instruction data to the body surface stimulation unit 16 (the electric acupuncture device 26 in Example 1). As a result, the body surface stimulation unit 16 is made to output an electric signal in conformity with the initial value of stimulation condition.

At the step S2, the body surface stimulation regulation unit 14 outputs the stimulation condition datum as an instruction data to the body surface stimulation unit 16. The body surface stimulation regulation unit 14 also acquires the vital data (the pulse rate in Example 1) output from the vital reaction quantitative evaluation unit 12 (the electrocardiographic meter 22 in Example 1) in accordance with the stimulation condition datum.

At the steps S3-S6, the body surface stimulation regulation unit 14 outputs stimulation condition candidate values 1-4 formed by performing a specific calculation on the stimulation condition datum as instruction data to the body surface stimulation unit 16. Incidentally, as examples of the “specific calculation,” the act of increasing the stimulation condition datum by several tens of % and the act of decreasing the stimulation condition datum by several tens of % may be cited, though not exclusively. The body surface stimulation regulation unit 14 also acquires the vital data output by the body reaction quantitative evaluation means 12 in accordance with the stimulation condition candidate values 1-4.

At the final step S6, the body surface stimulation regulation unit 14 searches the optimum vital data most suitable for the purpose of therapy from the plurality of vital data acquired at the steps S2-S6 and sets the stimulation condition datum or the stimulation condition candidate values 1-4 that have been output at the time of acquisition of the optimum vital data as new stimulation condition datum.

From this point forward, the process of the steps S2-S6 is repetitively carried out till the vital data reaches the value that befits the purpose of therapy.

Though the example depicted in FIG. 5 (A) has four stimulation condition candidate values, the searching control does not need to limit the number of these values to 4 but may change it to less than 4 or not less than 5. For the purpose of securing the quality of real time in the searching control, however, the number of the stimulation condition candidate values is preferred to be within 10.

Though the duration of outputting the electric signal conforming to the stimulation condition datum and the stimulation condition candidate value from the body surface stimulation unit 16 does not need to be particularly limited, the duration of outputting the stimulation condition datum and the stimulation condition candidate value is preferred to be fixed for the purpose of simplifying the process of searching control.

Further, the stimulation that is controlled by the searching control is not limited to one kind. The searching control may be applied as illustrated in FIG. 5 (B), for example, to a plurality of kinds of stimulation (three kinds in this case). Incidentally, in this case, the searching control may be applied to one kind of stimulation sequentially selected from the plurality of kinds of stimulation or the searching control may be applied at once to all the plurality of kinds of stimulation.

Back to FIG. 4, this experiment started the therapy (electric simulation) at the point one minute after starting the measurement of the pulse rate and gave a stimulation voltage 1V and a stimulation frequency 1 Hz as the initial values of stimulation condition.

Then, the voltage regulation at a fixed frequency was carried out at intervals of one minute for the duration of 3 minutes. The voltage was increased to 1.2 times the fixed value when the decrease of pulse rate aimed at by the therapy was observed.

Then, the frequency regulation at a fixed voltage (fixed at the preceding last voltage) was carried out at intervals of one minute for the duration of 15 minutes. With the frequency reckoned in sets each of 3 minutes, one-minute stimulations were sequentially carried out in the order of basic frequency (corresponding to the stimulation condition datum)→the basic frequency increased by 10% (corresponding to the stimulation condition candidate value)→the basic frequency decreased by 10% (corresponding to the stimulation condition candidate value). Immediately thereafter, the mean values of the pulse rates in the three stimulation conditions (basic frequency, basic frequency increased by 10%, and basic frequency decreased by 10%) were compared in search of the lowest pulse rate (the vital data most suitable for a decrease of the pulse rate aimed at by the therapy). Then, the stimulation condition datum or the stimulation condition candidate value that corresponds to the lowest pulse rate was decided and used as the basic frequency of the next set. This procedure was repeated to a total of 5 sets.

Next, with the voltage fixed (at the preceding final voltage), the regulation of voltage was carried out every minute for the duration of 15 minutes. With the voltage reckoned in sets each of 3 minutes, one-minute stimulations were sequentially carried out in the order of basic voltage (corresponding to the stimulation condition datum)→the basic voltage increased by 10% (corresponding to the stimulation condition candidate value)→the basic voltage decreased by 10% (corresponding to the stimulation condition candidate value). Immediately thereafter, the mean values of the pulse rates in the three stimulation conditions (the basic voltage, the basic voltage increased by 10%, and the basic voltage decreased by 10%) were compared in search of the lowest pulse rate (the vital data most suitable for a decrease of the pulse rate aimed at by the therapy). Then, the stimulation condition datum or the stimulation condition candidate value corresponding to the lowest pulse rate was decided and used as the basic voltage for the next set. This procedure was repeated to a total of 5 sets.

Thereafter, the voltage-fixed frequency regulation (15 minutes) and the frequency-fixed voltage regulation (15 minutes) were alternately repeated.

As a result, the experiment succeeded in stably and infallibly lowering the pulse rate by the therapy with the apparatus for body surface stimulation treatment 20 from the level of about 315 (beats/min) existing prior to the therapy to the level of 300 (beats/min) or to below this level.

FIG. 6 illustrates the states of changes of blood pressure and sympathetic nerve activity that were induced by the electric acupuncture device 26 in the case (A) of imparting an electric stimulation of low relative frequency (frequency 1 Hz and voltage 2 V), in the case (B) of imparting an electric stimulation of medium relative frequency (frequency 10 Hz and voltage 2 V), and in the case (C) of imparting an electric stimulation of high relative frequency (frequency 100 Hz and voltage 2 V).

As clearly noted from FIG. 6, the vital response to the body surface stimulation was much complicated and revealed intense time variation. While it is, therefore, difficult to have the industrial algorithm that is not directed to the vital signal control diverted per se to the apparatus for body surface stimulation treatment, the apparatus for body surface stimulation treatment 20 that has adopted searching control enables the vital data obtained by the vital reaction quantitative evaluation unit to approximate expeditiously and precisely the optimum vital data most suitable for the purpose of therapy and also enables the effect of therapy to be further exalted.

Since the apparatus for body surface stimulation treatment 20 that is involved in Example 1 is configured by being furnished with a vital reaction quantitative evaluation unit (the electrocardiographic meter 22 in Example 1) capable of measuring, determining and evaluating the vital reaction of a patient and outputting the measured, determined and evaluated vital reaction as the vital data, a body surface stimulation regulating means (the regulating device 24 in Example 1) capable of comparing the vital data obtained by the vital reaction quantitative evaluation unit with the vital data aimed at by the therapy, regulating the stimulation properties to be imparted to the body surface tissue of the patient in accordance with the result of the comparison, and outputting the regulated stimulation properties as an instruction data, and a body surface stimulation unit (the electric acupuncture device 26 in Example 1) capable of physically or chemically stimulating the body surface tissue of the patient based on the instruction data obtained by the body surface situation regulation means, it is enabled by the vital reaction quantitative evaluation unit, the body surface stimulation regulation unit, and the body surface stimulation unit to implement the regulation of the body surface stimulation in the form of a closed loop and continuously. Thus, it can overcome the uncertainty of the conventional body surface stimulation treatment and manifest a stable therapeutic effect.

Incidentally, though the autonomic nerve activity (sympathetic nerve activity and vagus nerve activity), unlike such symptoms as pain, does not necessarily mount up to a subjective symptom, it is extremely important for the therapy of pathologic aggravation. Since, the autonomic nerve reacts to any of the body surface stimulations in several seconds to several minutes, however, it is immensely complicated and difficult for a human being to regulate the body surface stimulation manually. In this respect, the apparatus for body surface stimulation treatment 20 is enabled to regulate the autonomic nerve not manually but automatically by causing the regulation of the body surface stimulation to proceed in the form of a closed loop and continuously.

Then, it is generally known that the increase of heart rate reflects an increase of the sympathetic nerve activity and a decrease of vagus nerve activity and the decrease of the heart rate reflects a decrease of the sympathetic nerve activity and an increase of the vagus nerve activity. The frequency analysis of the electrocardiographic wave form (the Fourier transform method, the autoregression method, the complex demodulation method, etc.) enables acquisition of the indexes of sympathetic nerve activity and vagus nerve activity. In this respect, since the apparatus for body surface stimulation treatment 20 is furnished with the electrocardiographic meter 22 as a vital reaction quantitative evaluation unit, it is capable of evaluating the autonomic nerve activity by measuring heart rate and electrocardiographic wave form.

Further, since the body surface stimulation unit is an electric stimulation means for electrically stimulating the body surface tissue of a patient, it is enabled by the electric stimulation to improve such autonomic abnormalities as, for example, chronic cardiac failure and ischemic heart disease, namely an abnormal increase of the sympathetic nerve activity or an abnormal decrease of the vagus nerve activity.

Moreover, since the body surface stimulation regulation unit is so configured as to allow regulation of at least one of the factors of voltage, electric current, frequency, frequency of stimulation, and duration of stimulation by the electric stimulation means, it is enabled to vary the influence exerted on the patient and perform therapy of high precision.

Incidentally, the apparatus for body surface stimulation treatment involved in this invention is not limited to the configuration of the apparatus for body surface stimulation treatment 20 contemplated by Example 1. It may have the electrocardiographic meter 22, the regulation device 24, and the electric acupuncture device 26 severally furnished with a radio means, as illustrated in FIG. 7, for example, and enabled to transmit and receive the data of each of the devices to be effected in a wireless manner.

When the body surface stimulation regulation unit is so configured as to enable acquisition of the vital data via radio communication from the vital reaction quantitative evaluation unit and the body surface stimulation unit as well is so configured as to enable acquisition of the instruction data via radio communication from the body surface stimulation regulation unit, it is made possible to prevent and cure such diseases of the circulatory organs as arrhythmia and abnormality of circulatory kinetic and accomplish therapy in case of emergency by radio transmitting the vital data from the vital reaction quantitative evaluation unit (such as, for example, electrocardiographic meter, local continuous blood flow meter, blood pressure meter, or neuroelectric meter) to the body surface stimulation regulation unit (such as, for example, a personal computer) installed in a medical organ such as a hospital located at a remote district and remote handling the body surface stimulation regulation unit (such as, for example, an electric acupuncture device) via radio communication thereby effecting therapy of autonomic nerve activity or heart rate. The lethal arrhythmia during the onset of myocardial infarct that occurs at a place like a patient's own home other than a hospital that is difficult to cure by the existing medical care can be cured and prevented.

While Example 1 treated the vital reaction quantitative evaluation unit, the body surface stimulation regulation unit, and the body surface stimulation unit as mutually independent components, this invention does not need to be limited to this principle. It may have the regulation device 24 and the electric acupuncture device 26 integrated as illustrated in FIG. 8, for example. In this case, the apparatus for body surface stimulation treatment is enabled to gain in convenience because it can be transported and handled with ease. As a matter of course, the electrocardiographic meter 22 and the regulation device 24 may be integrated or the electrocardiographic meter 22, the regulation device 24, and the electric acupuncture device 16 may be wholly integrated.

Further, though Example 1 elected adoption of the electrocardiographic meter 22 as the vital reaction quantitative evaluation unit 12 mentioned above, this invention does not need to have the vital reaction quantitative election means limited thereto. It may adopt a local continuous blood flow meter, a blood pressure meter (to be explained in Example 2), a neuroelectric meter, and an electrogastric meter (the same applies to Examples 2-5).

Further, in Example 1, the body surface stimulation treatment device utilizing the searching control mentioned above was made to serve as a body surface stimulation regulation unit. The algorithm for regulating the body surface stimulation regulation unit, however, does not need to be limited thereto. Such industrial control algorithms as PID control, fuzzy control, and neural network may be converted or applied instead (the same applies to Examples 2-5).

### Example 2

Now, a apparatus for body surface stimulation treatment 30 involved in Example 2 of this invention will be explained in detail below.

FIG. 9 is a drawing illustrating in type section the apparatus for body surface stimulation treatment 30 that is involved in Example 2 of this invention and FIG. 10 is a block diagram illustrating the main configuration of the body surface stimulation treatment device 30.

This apparatus for body surface stimulation treatment 30 uses a blood pressure meter 32 capable of measuring pulse rate and blood pressure as vital signals reflecting changes in visceral functions of a patient 28 in the place of the electrocardiographic meter 22 of the apparatus for body surface stimulation treatment 20 involved in the preceding Example 1. Since the rest of the configuration is identical with the configuration of the apparatus for body surface stimulation treatment 20 involved in the preceding Example 1, the identical component parts in the drawing will be denoted by the identical signs and the explanations thereof will be omitted.

This apparatus for body surface stimulation treatment 30 compares such vital data as pulse rate and blood pressure measured, determined and evaluated by the blood pressure meter 32 with the vital data (such as, for example, adequate pulse rate and blood pressure) aimed at by the relevant therapy, regulates the properties (such as, for example, voltage, electric current, frequency, frequency of stimulation, and duration of stimulation) of the stimulation (electric stimulation in this case) to be imparted to the tissue of the body surface (foot in this case) of the patient 28 in conformity with the result of the comparison, and transmits the regulated stimulation properties as an instruction data to the electric acupuncture device 26. The electric acupuncture device 26 implements the relevant therapy by electrically stimulating the body surface tissue of the patient 28 in accordance with the instruction data received from the regulation device 24. By causing the changes of blood pressure and pulse rate of the patient 28 caused by the electric stimulation to be measured, determined and evaluated again with the blood pressure meter 32, it is made possible to form a closed loop of blood pressure meter 32→regulation device 24→electric acupuncture device 26 and appropriate the blood pressure and the pulse rate of the patient 28.

FIG. 11 shows the results of an experiment performed by the present inventor using the apparatus for body surface stimulation treatment 30. Incidentally, FIG. 11 is a graph showing the relation between the values of blood pressure determined with the blood pressure meter 32 of the apparatus for body surface stimulation treatment 30 and the voltage (stimulation voltage) and the frequency (stimulation frequency) of the electric signal emitted from the electric acupuncture device 26.

In this experiment, i.e., an animal (rabbit) test under the control of venous anesthesia and artificial respiration, the body blood pressure was continuously measured with a pressure catheter inserted through the right femoral artery and the electrocardiogram was measured as well with the electrocardiographic meter 32 and the pulse rate was monitored and evaluated in real time based on the consequently obtained results.

The outer side of the front face about 5 mm below the left stile bone (corresponding to the pit called “Ashi Sanri” in the Human Oriental Medicine) and the outer side of the front face about 1 cm above the articulation of foot were electrically stimulated by the electric acupuncture device 26 through the medium of acupuncture electrodes 26A (made of stainless steel) inserted to a depth of about 8 mm in the relevant outer sides.

The electric acupuncture device 26, by being externally controlled with the DA signal from a personal computer serving as the adjusting device 24, was enabled to effect automatic regulation of the stimulation voltage and the stimulation frequency in conformity with the actual evaluation value of pulse rate so as to lower the blood pressure. Incidentally, the regulation of the stimulation voltage and the stimulation frequency was carried out by means of the Algorithm 1 (searching control) described above.

More specifically, the therapy was started at the time one minute subsequent to the start of measurement of the blood pressure as illustrated in FIG. 11 and made to register a stimulation voltage of 0.8 V and a stimulation frequency of 1 Hz as the initial values of the stimulation conditions.

Then, the voltage regulation at a fixed frequency was carried out every minute for a duration of 7 minutes. The voltage was increased to 1.5 times the original value when the decrease of blood pressure aimed at by the therapy was observed.

The voltage regulation at a fixed frequency (fixed at the former final frequency) was carried out every minute for duration of 15 minutes. With the voltage reckoned in sets each of 3 minutes, one-minute stimulations were sequentially carried out in the order of basic voltage (corresponding to the stimulation condition datum)→the basic voltage increased by 10% (corresponding to the stimulation condition candidate value)→the basic voltage decreased by 10% (corresponding to the stimulation condition candidate value). Immediately thereafter, the mean values of the blood pressure in the three stimulation conditions (basic voltage, basic voltage increased by 10%, and basic voltage decreased by 10%) were compared in search of the lowest pulse rate (the vital data most suitable for a decrease of the blood pressure aimed at by the therapy). Then, the stimulation condition datum or the stimulation condition candidate value that corresponded to the lowest blood pressure was decided and used as the basic voltage of the next set. This procedure was repeated to a total of 5 sets.

Thereafter, the regulation of voltage-fixed frequency (15 minutes) and the regulation of frequency-fixed voltage (15 minutes) were alternately carried out repetitively.

As a result, the therapy performed by using the apparatus for body surface stimulation treatment 30 succeeded in enabling the blood pressure showing a high value of about 150 (mmHg) prior to the therapy to be continuously lowered by 20-30 (mmHg).

Further, the present inventor performed another experiment using the apparatus for body surface stimulation treatment 30. The results of this experiment are shown in FIG. 12. FIG. 12 (A) is a graph showing the relation between the value of blood pressure measured with the blood pressure meter 32 of the apparatus for body surface stimulation treatment 30 at the times about 1 minute-12 minutes subsequent to the measurement of blood pressure and the frequency (stimulation frequency) of the electric signal output from the electric acupuncture device 26 and FIG. 12 (B) is a graph showing the state existing between the points of the 6th minute-8th minute of the graph of FIG. 12 (B) in an enlarged scale.

In this experiment, the digital arteria of a man maintained in arousal consciousness was continuously measured by the low stress cuff pressure load method.

The outer side of the front face about 9 cm below the left stile bone (corresponding to the pit called “Ashi Sanri” in the Human Oriental Medicine) and the outer side of the front face about 10 cm above the articulation of foot were electrically stimulated by the electric acupuncture device 26 through the medium of an acupuncture electrodes 26A (made of stainless steel) inserted to a depth of about 2 cm in the relevant outer sides.

The electric acupuncture device 26 was externally controlled by the DA signal from a personal computer serving as the regulation device 24 and the stimulation frequency was automatically regulated in conformity with the actual value of blood pressure so as to lower the blood pressure. Incidentally, the regulation of this stimulation frequency was performed with the Algorithm 2 (adaptive control) that will be explained below.

FIG. 13 is a graph showing an example of the application of the “adaptive control” contemplated by this invention to the control of electric stimulation.

When the vital function and signal reacts biphasically to a stimulation in the apparatus for body surface stimulation treatment 30 having applied the adaptive control, the body surface stimulation regulation unit (the regulation device 24 in Example 2) outputs an instruction data in case the vital data obtained from the vital reaction quantitative evaluation unit (the blood pressure meter 32 in Example 2) fits the purpose of the therapy and stops the outputting of the instruction data in case the vital data obtained from the vital reaction quantitative evaluation unit does not fit the purpose of the therapy. When the therapy directed to lowering blood pressure is carried out as illustrated in FIG. 13, for example, the blood pressure is measured at intervals of proper length for the purpose of avoiding a blood pressure increasing reaction and inducing only a blood pressure decreasing reaction by continuing the stimulation when the present blood pressure is lower than the previously measured blood pressure and stopping the stimulation when the present blood pressure is higher than the previously measured blood pressure.

So long as the body surface stimulation regulation unit is so configured that the instruction data is output when the vital data obtained from the vital reaction quantitative evaluation unit fits the purpose of therapy and the outputting of the instruction data is stopped when the vital data obtained from the vital reaction quantitative evaluation unit does not fit the purpose of the therapy, it enables the vital data obtained from the vital reaction quantitative evaluation unit to approximate the state most suitable for the purpose of therapy, though the control to be involved is simple and easy.

Back to FIG. 12, this experiment performed electric stimulation for durations of 30 seconds as one set and passed it through a real time digital low pass filter for 10 seconds, as well evaluated blood pressure with a mean value of 3 seconds, continued the stimulation for the next 3 seconds when the present value of blood pressure was higher than the previous value of blood pressure, and stopped the stimulation for the next 3 seconds when the present value of blood pressure is lower than the previous value of blood pressure. It further performed searching regulation of the frequency of the electric stimulation every duration of 30 seconds.

As a result, the therapy using the apparatus for body surface stimulation treatment 30 succeeded in continuing the decrease of blood pressure by 20-30 (mmHg), though the blood pressure prior to the therapy showed such a high value as about 150 (mm Hg).

As described above, the increase of pulse rate reflects an increase of sympathetic nerve activity and a decrease of vagus nerve activity and the decrease of pulse rate reflects a decrease of sympathetic nerve activity and an increase of vagus nerve activity. Further, the frequency analysis (Fourier transform method, autoregressive method, complex demodulation method, etc.) of the blood pressure wave form generally enables acquisition of the indexes of sympathetic nerve activity and vagus nerve activity. Thus, the apparatus for body surface stimulation treatment 30 involved in Example 2 enables the autonomic nerve activity to be evaluated by measuring pulse rate and blood pressure (wave form).

Particularly, a patient in the morbidity (myelic injury, for example) that lacks vital blood pressure maintaining function (pressure reflex function) has his daily life restricted because he is unable to assume an arousal posture due to severe orthostatic hypotension. He is, however, enabled by the apparatus for body surface stimulation treatment 30 involved in Example 2 to receive a therapy for maintaining blood pressure. Since the cure with a drug spends time in the manifestation or extinction of its effect, the adjustment of blood pressure is extremely difficult during continuous change of posture such as lying position, standing position or lying position. The apparatus for body surface stimulation treatment 30 involved in Example 2, however, is capable of curing blood pressure in such a morbidity because it enables the electric acupuncture device 26 allowing quick manifestation and extinction of effect to be automatically regulated continuously.

Though the preceding Example 1 and the present Example 2 both adopted the electric acupuncture device 26 as the body surface stimulation unit mentioned above, the body surface stimulation unit contemplated by this invention does not need to be limited thereto. This invention allows adoption of an endermic electric nerve stimulation device (a device for imparting electric stimulation to the nerve), an endermic electric muscle stimulation device (a device for imparting electric stimulation to the muscle), and an endermic electric skin stimulation device (a device for imparting electric stimulation to the skin), for example, besides the electric acupuncture device 26.

Further, the apparatus for body surface stimulation treatment involved in this invention is not limited to the configuration of the apparatus for body surface stimulation treatment 30 that is involved in the present Example 2. For example, the blood pressure meter 32, the regulation device 24, and the electric acupuncture device 26 may be severally furnished with a wireless means and adapted to transmit and receive data to and from each of the component by wireless communication.

Furthermore, though the present Example 2 handled the vital reaction quantitative evaluation unit, the body surface stimulation regulation unit, and the body surface stimulation unit independently of one another, this invention does not need to be limited to this mode of embodiment. For example, the regulation device 24 and the electric acupuncture device may be integrated as illustrated in FIG. 15. Naturally, the blood pressure meter 32 and the regulation device 24 may be integrated or the blood pressure meter 32, the regulation device 24, and the electric acupuncture device 26 may be wholly integrated.

### Example 3

Now, a apparatus for body surface stimulation treatment 40 involved in Example 3 of this invention will be explained in detail below.

FIG. 16 is a drawing illustrating in type section the apparatus for body surface stimulation treatment 40 involved in Example 3 of this invention and FIG. 17 is a block diagram illustrating the main configuration of the apparatus for body surface stimulation treatment 40.

This apparatus for body surface stimulation treatment 40 adopts an air conditioner device 42, which is one of the thermal stimulation unit for physicochemically stimulating the body surface tissue of a patient by dint of changes of heat of cooling and atmospheric pressure, in the place of the electric acupuncture device 26 of the apparatus for body surface stimulation treatment 20 involved in the preceding Example 1 and enables wireless intercommunication between the regulation device 24 and the air conditioner 42. Since the rest of the configuration is identical with the configuration of the apparatus for body surface stimulation treatment 20 involved in the preceding Example 1, the identical component parts will be denoted by the identical signs and the explanations thereof will be omitted.

This apparatus for body surface stimulation treatment 40 compares such vital data as pulse rate and blood pressure measured, determined and evaluated by the electrocardiographic meter 22 with the vital data (such as, for example, adequate pulse rate and blood pressure) aimed at by the relevant therapy, regulates the properties (such as, for example, the set temperature, the set humidity, wind force, and the switching of warming and cooling of the air conditioner device 42) of the stimulation (physicochemical stimulation in this case) to be imparted to the tissue of the body surface of the patient 28 in conformity with the result of the comparison, and transmits the regulated stimulation properties as an instruction data to the air conditioner device 42. The air conditioner device 42 implements the relevant therapy by imparting thermal stimulation to the patient 28 in accordance with the instruction data received from the regulation device 24. By causing the changes of blood pressure and pulse rate of the patient 28 induced by the physicochemical stimulation to be measured, determined and evaluated again with the electrocardiographic meter 22, it is made possible to form a closed loop of electrocardiographic meter 22→regulation device 24→air conditioner device 42 and appropriate the heart rate and the electrocardiographic wave form of the patient 28.

Generally, the ambient temperature is known to vary the autonomic nerve activity and the sympathetic nerve activity is inevitably strained no matter whether the room temperature is high or low. Since the emotion is degraded and the autonomic nerve activity is made sensitive to the ambient temperature particularly during the sleep as compared with the daytime, the control of the ambient temperature gains in effect and the patient is possibly made unable to alter the setting of the air conditioner by himself as well. Thus, in the case of the apparatus for body surface stimulation treatment 40 involved in the present Example 3, since it is furnished with a thermal stimulation unit (the air conditioner device 42 in this case) adapted to stimulate the body surface tissue of the patient physicochemically with changes of cooling and atmospheric pressure, such autonomic nerve abnormalities as, for example, chronic cardiac failure and ischemic heart disease, namely an abnormal increase of the sympathetic nerve activity and an abnormal decrease of the vagus nerve activity, can be improved by the physicochemical stimulation utilizing the changes of cooling and atmospheric pressure.

Incidentally, the algorithm 1 (searching control) described above fits the control of the air conditioner device 42 involved in the present Example 3. This fact rests on the following reason.

The sympathetic nerve activity is exalted no matter whether the ambient temperature is unduly low or unduly high, as illustrated in FIG. 18. When the Algorithm 1 is adopted for the control of the air conditioner device 42, however, the sympathetic nerve activity can be minimized (or the heart rate can be minimized) because the ambient temperature that minimizes the sympathetic nerve activity (or minimizes the heart rate) can be easily searched. The graph of FIG. 18 has resulted from actually measuring the nerve activity of a man by means of a minute neuroelectric meter.

By implementing the control in the closed loop of electrocardiographic meter 22→regulation device 24→air conditioner device 42 as depicted above, it is made possible to avoid the sympathetic nerve tension due to high ambient temperature and low ambient temperature and decrease the sympathetic nerve activity (simultaneously increase the vagus nerve activity) of a patient who is subjected to the therapy of cardiac failure during the sleep at night.

Incidentally, though the present Example 3 adopts the air conditioner device 42 as the body surface stimulation unit 16 mentioned above, the body surface stimulation unit contemplated by this invention does not need to be limited thereto. For example, such a device as a pediluvium device, a bathing device, or a sauna device that is capable of imparting thermal stimulation to the body surface may be adopted.

### Example 4

Now, an apparatus for body surface stimulation treatment 50 involved in Example 4 of this invention will be explained in detail below.

FIG. 19 (A) is a drawing illustrating in type section the apparatus for body surface stimulation treatment 50 involved in Example 4 of this invention and FIG. 20 is a block diagram illustrating the process for operating the apparatus for body surface stimulation treatment 50.

This apparatus for body surface stimulation treatment 50 adopts a massage device 52, which is one of the mechanical stimulation means for mechanically stimulating the body surface tissue of a patient, in the place of the electric acupuncture device 26 of the apparatus for body surface stimulation treatment 20 involved in the aforementioned Example 1 and as well causes the regulation device 24 to be disposed in the massage device 52. Since the rest of the configuration is identical with the configuration of the apparatus for body surface stimulation treatment 20 involved in the preceding Example 1, the identical component parts in the drawing will be denoted by the identical signs and the explanations thereof will be omitted.

This apparatus for body surface stimulation treatment 50 compares such vital data as heart rate and electrocardiographic wave form that have been measured, determined and evaluated by means of the electrocardiographic meter 22 with the vital data (such as, for example, appropriate heart rate and electrocardiographic wave form) aimed at by the therapy, regulates the properties (such as, for example, position of stimulation by the massage device 52, intensity of stimulation, frequency of stimulation, duration of stimulation, kind of stimulation <beating or rubbing>, etc.) of the stimulation (mechanical stimulation in this case) to be imparted to the tissue of the body surface (the back, the waist, and the feet in this case) of the patient 28 in conformity with the result of the comparison, and transmits the regulated properties of stimulation as an instruction data to the massage device 52. The massage device 52 carries out the therapy by mechanically stimulating the body surface tissue of the patient 28 based on the instruction data received from the regulation device 24. By causing the changes of heart rate and electrocardiographic wave form of the patient 28 induced by the mechanical stimulation to be measured, determined and evaluated again with the electrocardiographic meter 22, it is made possible to form a closed loop of electrocardiographic meter 22→regulation device 24→massage device 52 and adjust the heart rate and the electrocardiographic wave form of the patient 28.

In the case of the apparatus for body surface stimulation treatment 50 involved in the present Example 4, since it is furnished with the mechanical stimulation means (the massage device 52 in this case) for mechanically stimulating the body surface tissue of the patient 28, it is made possible by virtue of the mechanical stimulation to improve such autonomic nerve abnormalities as, for example, chronic cardiac failure and ischemic heart disease, namely an abnormal increase of the sympathetic nerve activity and an abnormal decrease of the vagus nerve activity.

Further, since the body surface stimulation regulation unit is so configured as to enable regulation of at least one of such factors as position of stimulation, intensity of stimulation, frequency of stimulation, and duration of stimulation that are assigned for the mechanical stimulation means, it is made possible to vary the influence exerted on the patient and accomplish therapy of high precision by regulating the mechanical stimulation properties in conformity with the vital reaction.

Incidentally, though the present Example 4 adopts the massage device 52 as the body surface stimulation unit 16 mentioned above, the body surface stimulation unit contemplated by this invention does not need to be limited thereto. For example, such a mechanical stimulation device as a manual pressure device or a truncus (such as cervical) traction device that mechanically stimulates the body surface tissue of a patient may be adopted. Also, a body surface stimulation treatment device 60 illustrated in FIG. 19 (B) that constitutes a massage device having built therein the vital reaction quantitative evaluation unit 12 and the electrocardiographic meter and the adjusting means 24 and causes the body surface stimulation regulation unit 12, the body surface stimulation regulation unit 14, and the body surface stimulation unit 15 mentioned above to be integrated.

### Example 5

Now, an apparatus for body surface stimulation treatment 70 involved in Example 5 of this invention will be explained in detail below.

FIG. 21 is a drawing illustrating in type section the apparatus for body surface stimulation treatment 70 involved in Example 5 of this invention and FIG. 22 is a block diagram illustrating the main configuration of the apparatus for body surface stimulation treatment 70.

This apparatus for body surface stimulation treatment 70 is configured by being furnished with a respiration monitor device 72 corresponding to the aforementioned vital reaction quantitative evaluation unit 12, the aforementioned regulation device 24, and an endermic electric skin stimulation device 74 corresponding to the aforementioned body surface stimulation unit 16. Incidentally, since the regulation device 24 is identical with the apparatus for body surface stimulation treatment 20 involved in the preceding Example 1, the explanation thereof will be omitted.

The respiration monitor device 72 involved in the present example is composed of a respiration flow meter 72A, an impedance prestimography 72B, and an endermic oxygen gas monitor 72C and is adapted to measure, determine and evaluate the respiration of an infant 76 and monitor the respiration (especially apnea).

The endermic electric skin stimulation device 74 imparts electric stimulation to the infant 76 via an electrode 74A that is pasted on the sole of the infant 76.

This apparatus for body surface stimulation treatment 70 compares the vital data (such as, for example, data concerning respiration including presence or absence of respiration, number of respirations, breathing capacity, and air volume per respiration) measured, determined and evaluated by the respiration monitor 72 with the vital data (such as, for example, appropriate number of respirations) aimed at by the therapy, regulates the properties (such as, for example, position of stimulation, intensity of stimulation, frequency of stimulation, and duration of stimulation assigned to the endermic electric skin stimulation device 74) of the stimulation (electric stimulation in this case) to be imparted to the tissue of the body surface (the sole in this case) of the infant 76 in conformity with the result of the comparison, and transmits the regulated stimulation properties as instruction data to the endermic electric skin stimulation device 74. The endermic electric skin stimulation device 74 implements the therapy by electrically stimulating the body surface tissue of the infant 76 based on the instruction data received from the regulation device 24. Then, by causing the change of respiration of the infant 76 due to the electric stimulation to be again measured, determined and evaluated with the respiration monitor 72, it is made possible to form a closed loop of respiration monitor 72→regulation device 24→endermic electric skin stimulation device 74 and adjust the respiration of the infant 76. The regulating device 26 may be so adapted as to emit an alarm sound when it happens to detect the apnea of the infant 76.

The apnea of the infant is a dangerous morbidity that leads hypoxia to death. Though all the cases of infant sudden death syndrome that are responsible for the annual death of 400-500 infants in Japan are not definitely ascribable exclusively to the apnea of infant, it is surmised that the arousal reaction, namely a defensive mechanism for recover from the apnea occurring during the sleep, is retarded by a certain reason (infancy, infection, constriction of the respiratory tract, etc.) and consequently suffered to aggravate the state of hypoxia, repress the respiration, induce vicious circle, and invite death finally. Meanwhile, it is empirically known that the infantile apnea is enabled by the skin stimulation (particularly the stimulation of the sole) to resume the respiration. It is, however, difficult for the man to control his respiration infallibly during his sleep at night. In this respect, the apparatus for body surface stimulation treatment 70 involved in the present Example 5 enables the respiration to be controlled not manually but automatically by regulating the body surface stimulation via a process performed in a closed loop and continuously.

Incidentally, though the present Example 5 adopts the endermic electric skin stimulation device 74 as the aforementioned body surface stimulation unit 16, this invention does not need to limit the body surface stimulation unit thereto. For example, the endermic electric nerve stimulation device or the endermic electric muscle stimulation device mentioned above may be used besides the endermic electric skin stimulation device 74. It is also allowable to adopt such a mechanical stimulation device as a massage device, a manual pressure device, or a truncus traction device.

Incidentally, though the present Example 5 adopts the respiration monitor device 72 composed of the respiration flow meter 72A, an impedance prestimography 72B, and a endermic oxygen gas monitor 72C as the vital reaction quantitative evaluation unit 12 mentioned above, this invention does not need to limit the vital reaction quantitative evaluation unit thereto. For example, a respiration gas concentration measuring device, an arterial blood oxygen carbon dioxide partial pressure measurement device, and a respiration monitor device capable of measuring various breathing kinetics (breathing capacity, number of respirations, amount of breathing air per respiration, changes of oxygen and carbon dioxide involved in respiration, and pulmonary compliance) may be adopted.

The apparatus for body surface stimulation treatment of this invention may be adopted for the therapy of man and animals.

### BRIEF DESCRIPTION OF DRAWINGS

The above and other objects, features and advantages of the present invention will become apparent from the following description and appended claims, taken in conjunction with the accompanying drawings.

FIG. 1 is a block diagram illustrating the main configuration of a apparatus for body surface stimulation treatment according to the mode of embodiment of this invention.

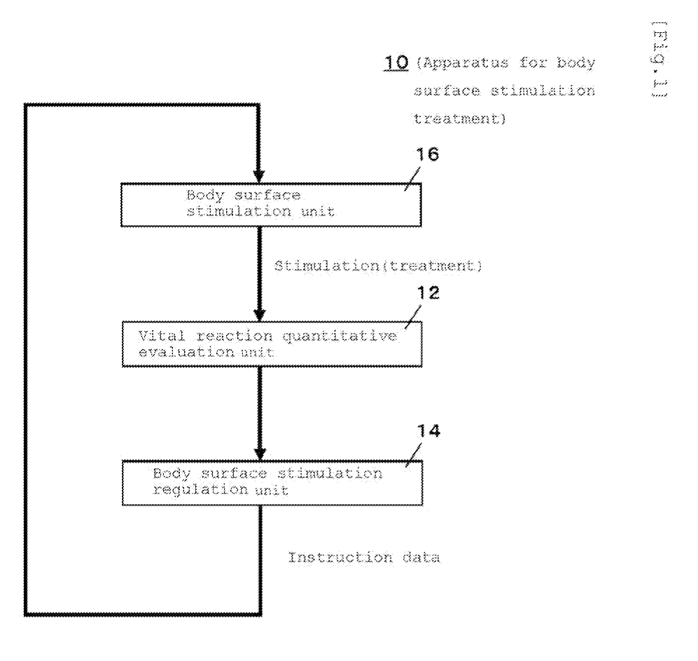

FIG. 2 is a drawing illustrating in type section the apparatus for body surface stimulation treatment according to Example 1 of this invention.

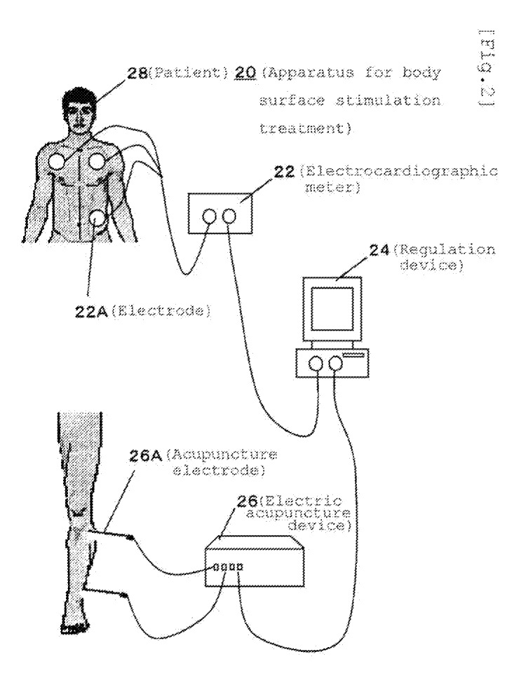

FIG. 3 is block diagram illustrating the main configuration of the apparatus for body surface stimulation treatment.

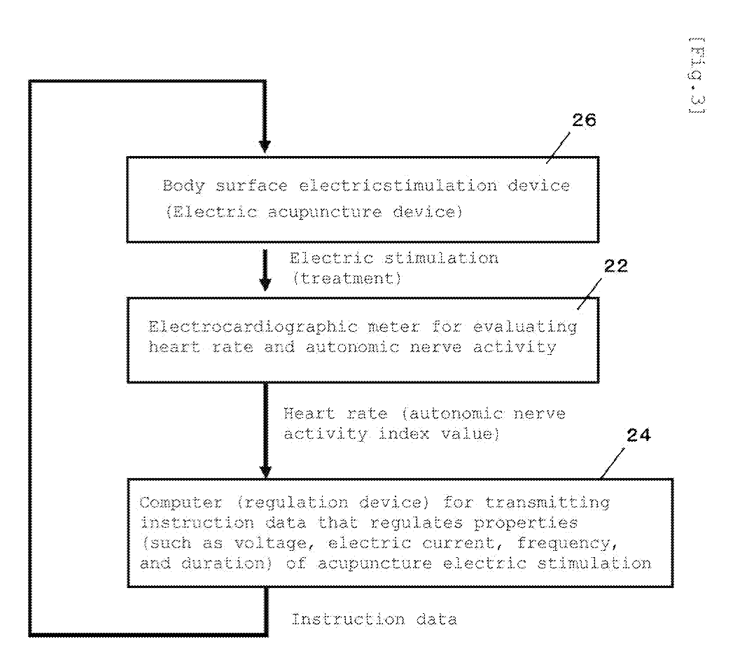

FIG. 4 is a graph illustrating the relation between the pulse rate measured by the electrocardiographic meter of the apparatus for body surface stimulation treatment and the voltage and the frequency output from an electric acupuncture device.

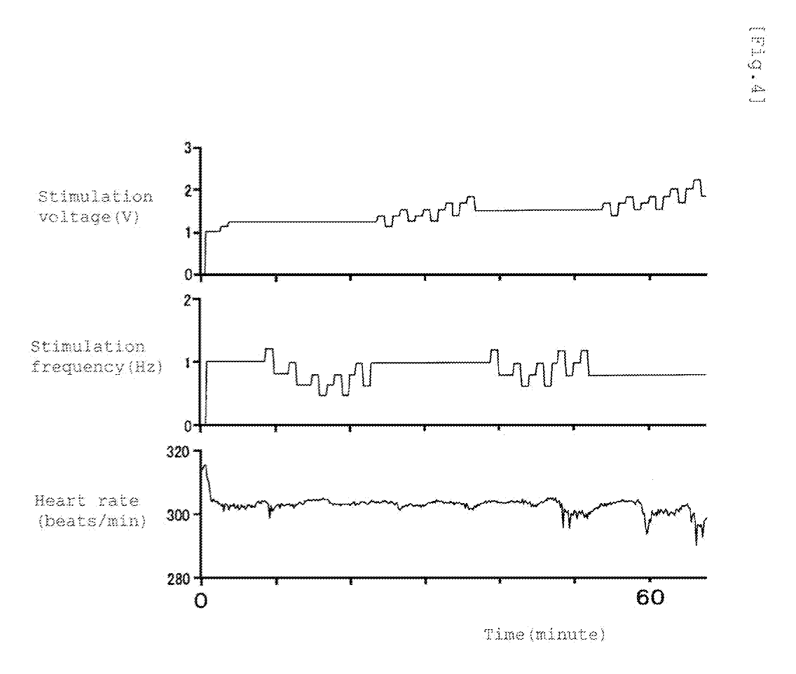

FIG. 5 (A) is a diagram illustrating in type section the flow of searching control of the apparatus for body surface stimulation treatment and (B) is a diagram illustrating the example of application of the searching control of the apparatus for body surface stimulation treatment.

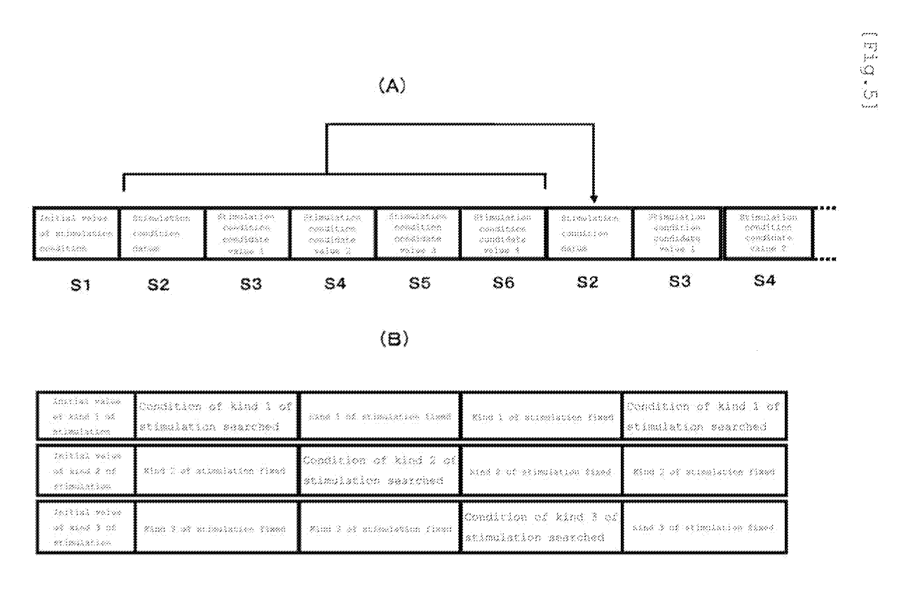

FIG. 6 is a graph illustrating the state of changes of blood pressure and sympathetic nerve activity in the case (A) of exerting an electric stimulation of low frequency, the case (B) of exerting an electric stimulation of medium frequency, and the case (C) of exerting an electric stimulation of high frequency by the electric acupuncture device of the apparatus for body surface stimulation treatment.

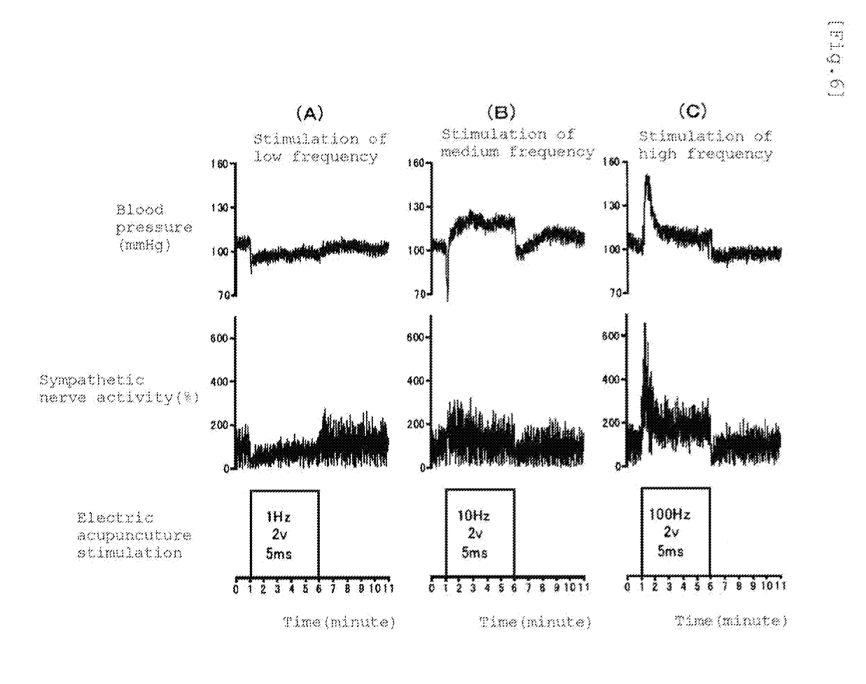

FIG. 7 is a drawing illustrating examples of applying radio communication to the apparatus for body surface stimulation treatment.

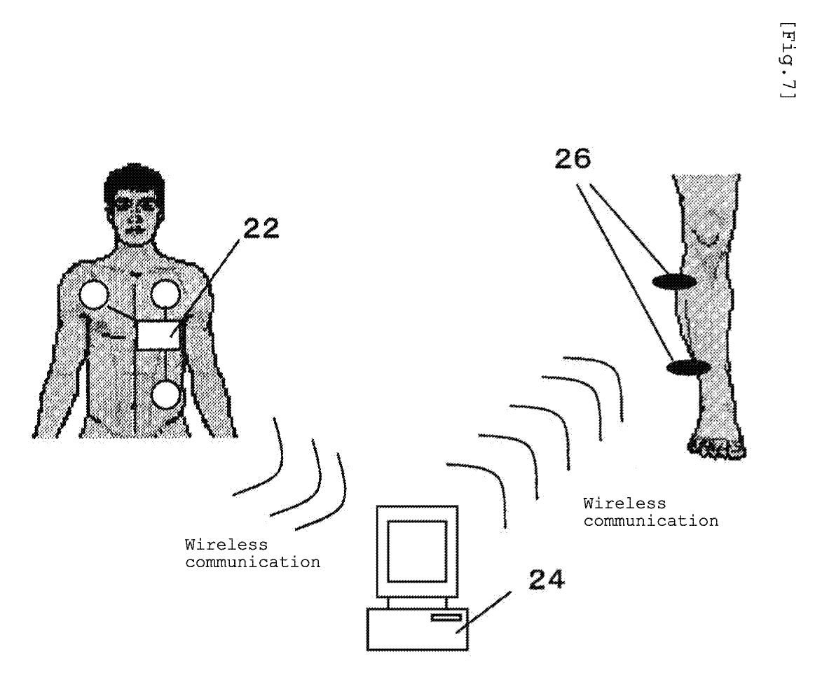

FIG. 8 is a drawing illustrating an example of integrating a regulating device and an electric acupuncture device of the apparatus for body surface stimulation treatment.

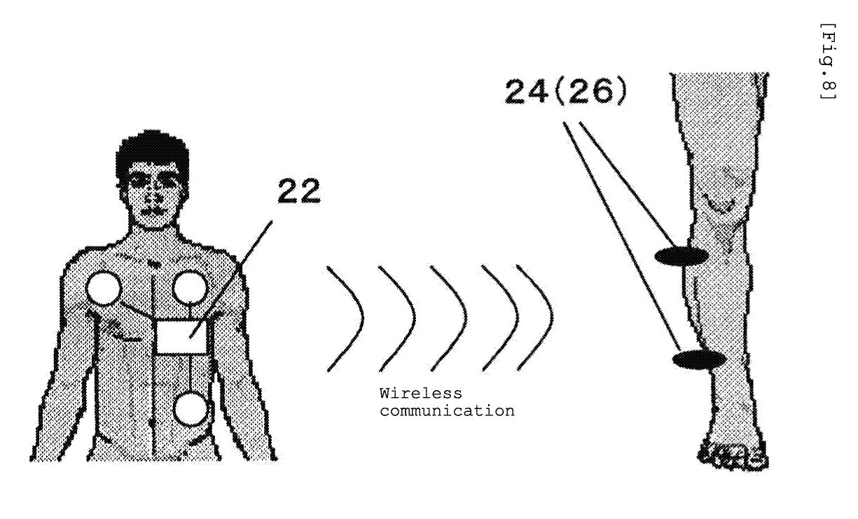

FIG. 9 is a drawing illustrating in type section a apparatus for body surface stimulation treatment according to Example 2 of this invention.

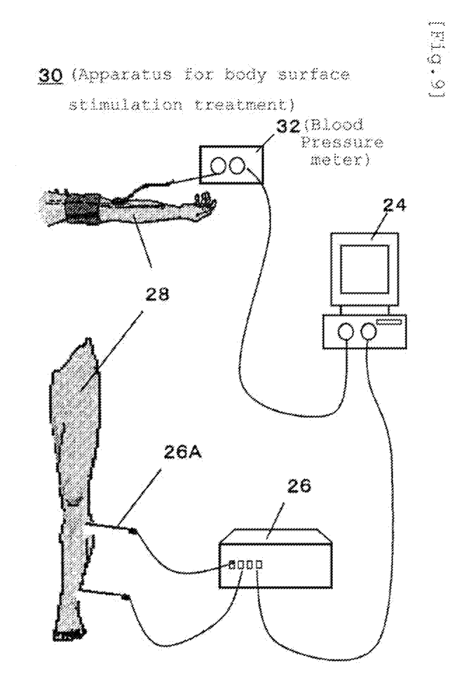

FIG. 10 is a block diagram illustrating the main configuration of the apparatus for body surface stimulation treatment.

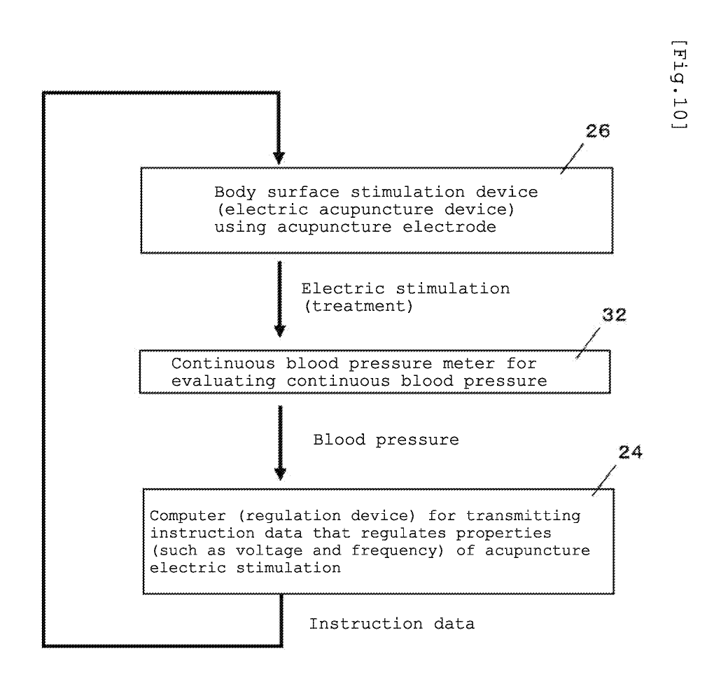

FIG. 11 is a graph illustrating the relation between the blood pressure value measured by the blood pressure meter of the apparatus for body surface stimulation treatment and the voltage and the frequency of an electric signal emitted from an electric acupuncture device.

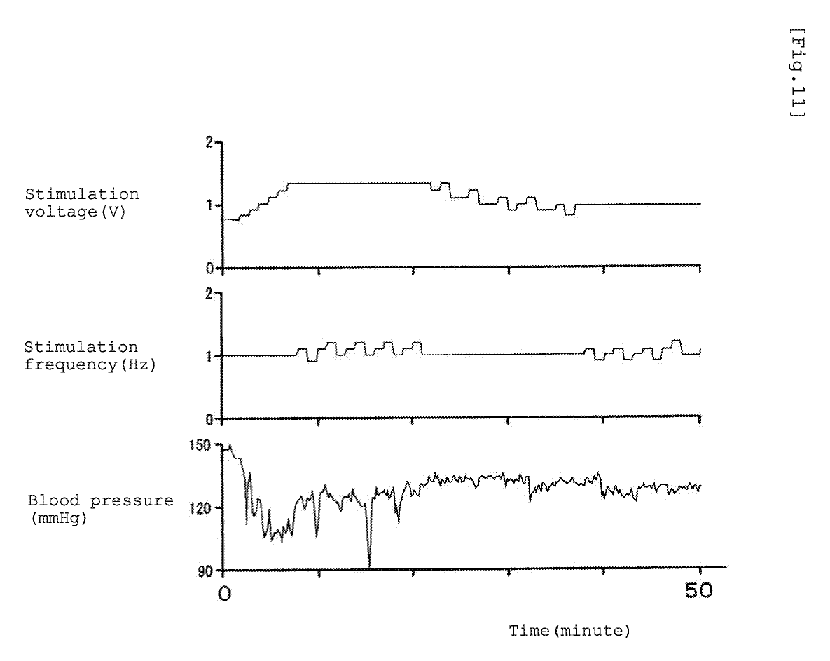

FIG. 12 (A) is a graph illustrating the relation between the blood pressure value measured by the blood pressure meter of the apparatus for body surface stimulation treatment (adaptive control) and the frequency of an electric signal emitted from the electric acupuncture device and (B) is a partly enlarged drawing of (A).

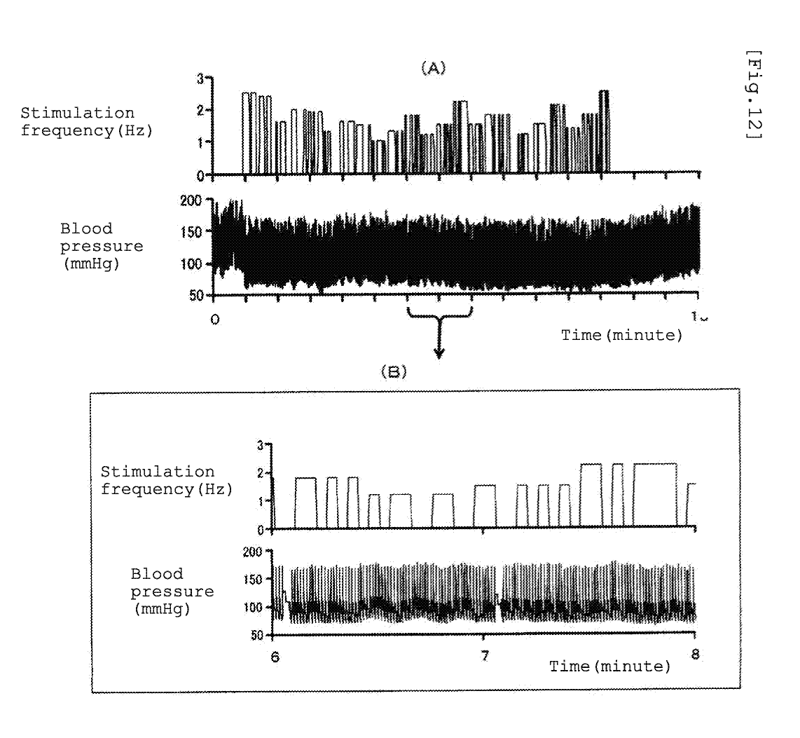

FIG. 13 is a graph illustrating an example of applying the adaptive control of the apparatus for body surface stimulation treatment to the control of an electric stimulation.

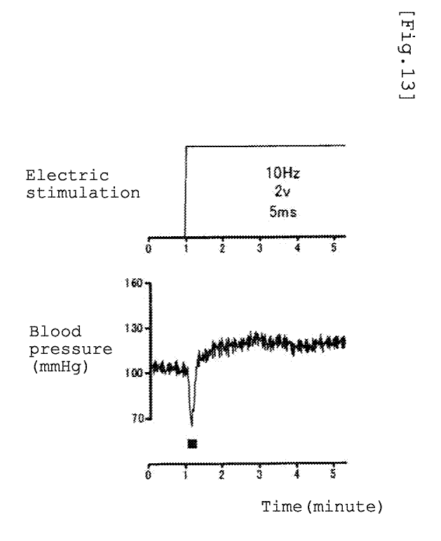

FIG. 14 is a drawing illustrating an example of applying radio communication to the apparatus for body surface stimulation treatment.

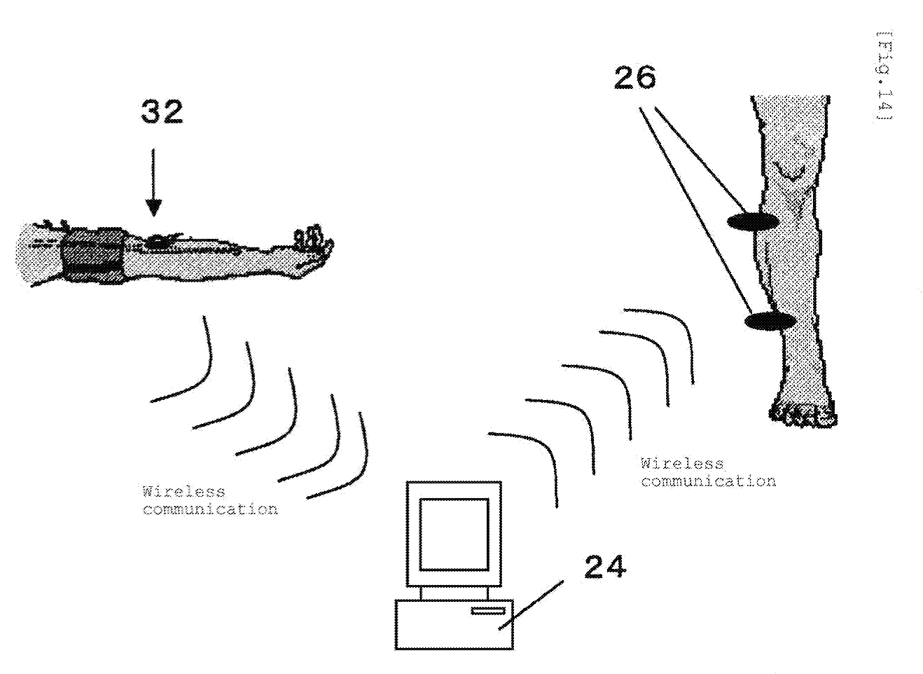

FIG. 15 is a drawing illustrating an example of integrating the control device and the electric acupuncture device of the apparatus for body surface stimulation treatment.

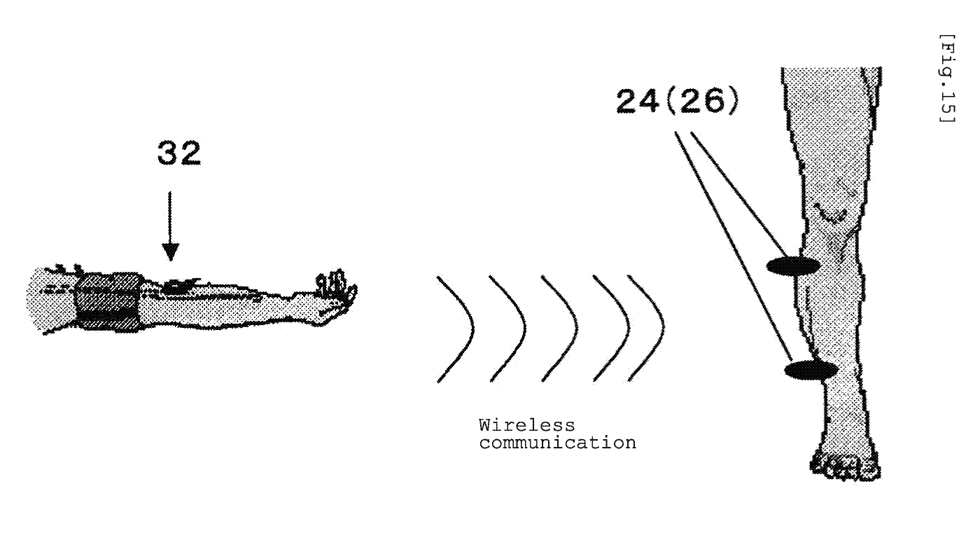

FIG. 16 is a drawing illustrating in type section the apparatus for body surface stimulation treatment according to Example 3 of this invention.

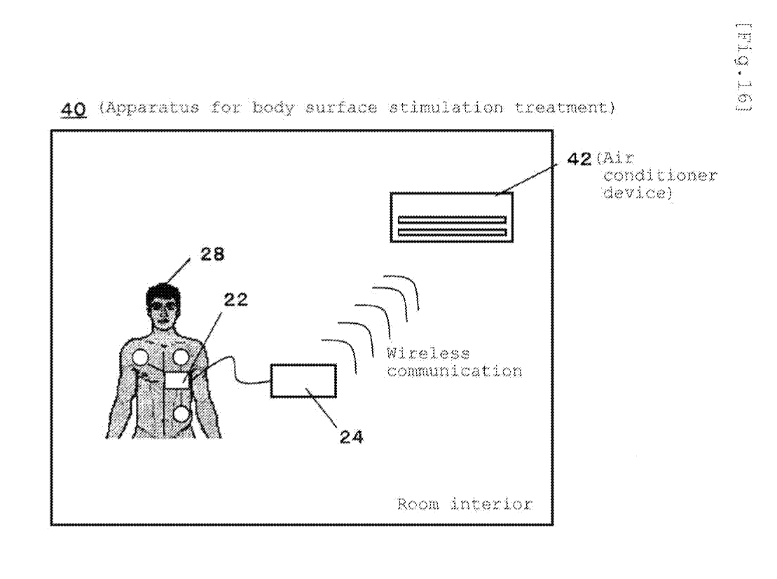

FIG. 17 is a block diagram illustrating the main configuration of the apparatus for body surface stimulation treatment.

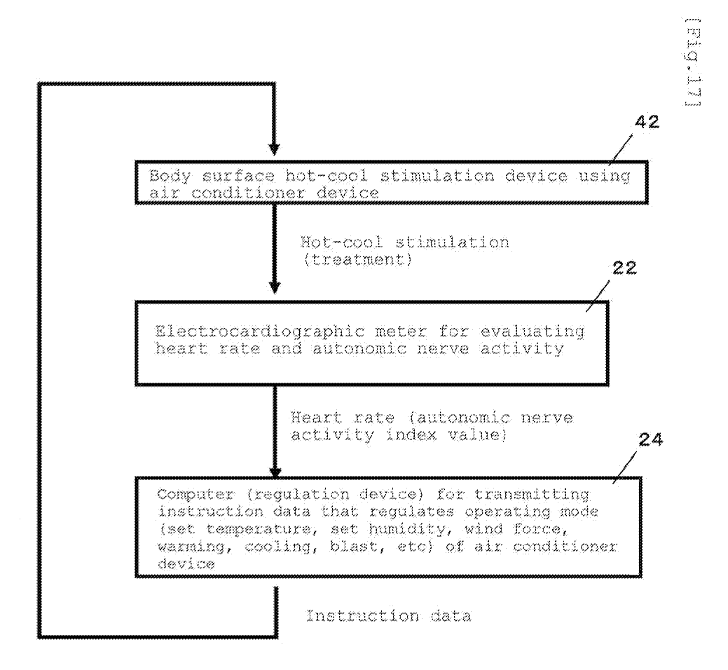

FIG. 18 is a graph illustrating the nerve activity of a man actually measured by the minute neuroelectric method.

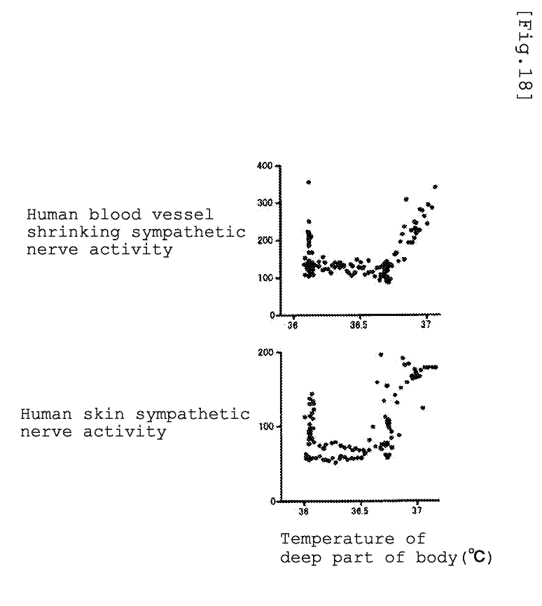

FIG. 19 (A) is a drawing illustrating in type section the apparatus for body surface stimulation treatment according to Example 4 of this invention and (B) is a drawing illustrating an example of integrating the electrocardiographic meter, the control device, and the massage device of the apparatus for body surface stimulation treatment.

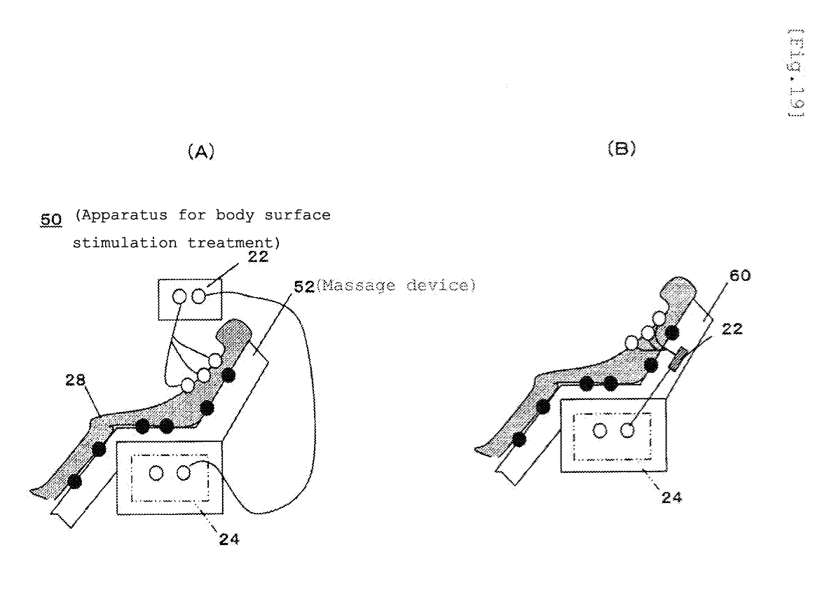

FIG. 20 is a block diagram illustrating the main configuration of the apparatus for body surface stimulation treatment.

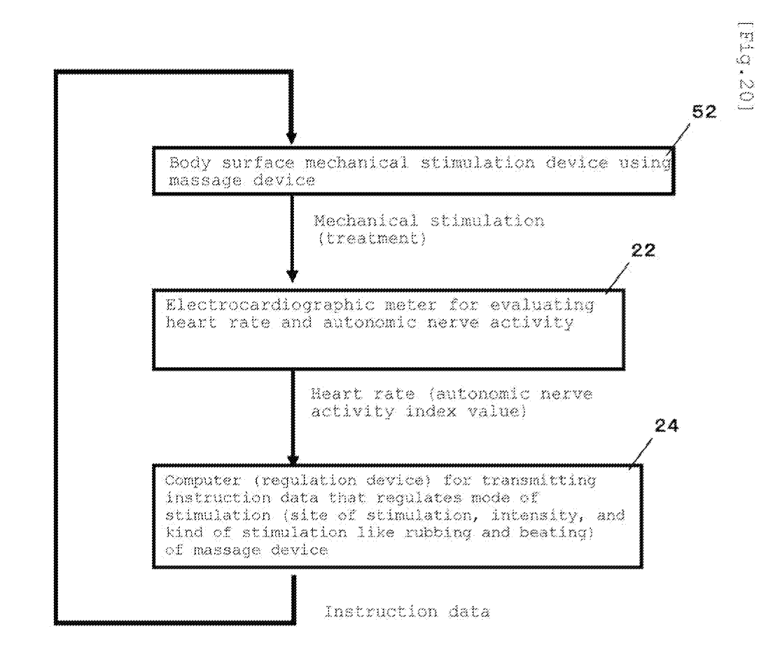

FIG. 21 is a drawing illustrating in type section the apparatus for body surface stimulation treatment according to Example 5 of this invention.

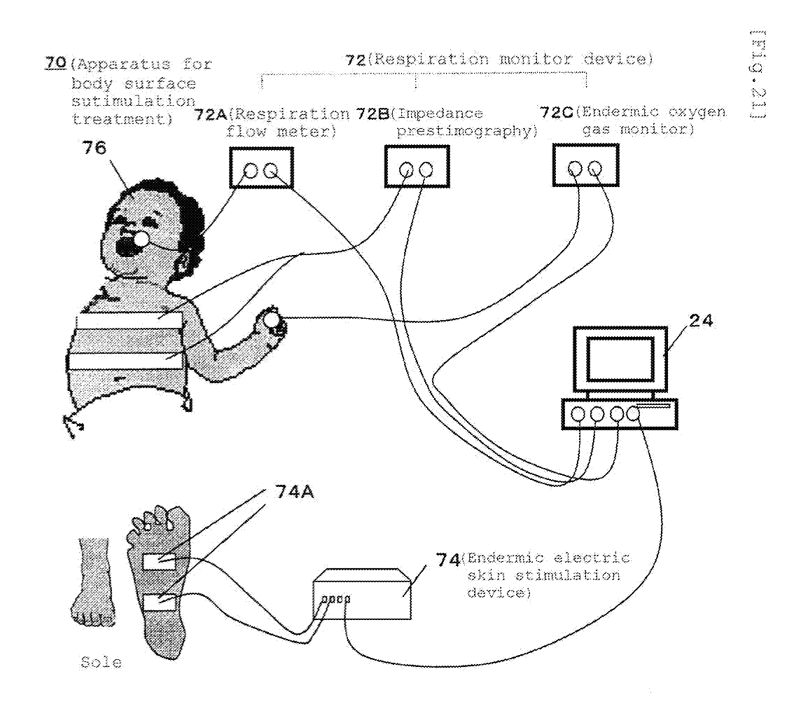

FIG. 22 is a block diagram illustrating the main configuration of the apparatus for body surface stimulation treatment.

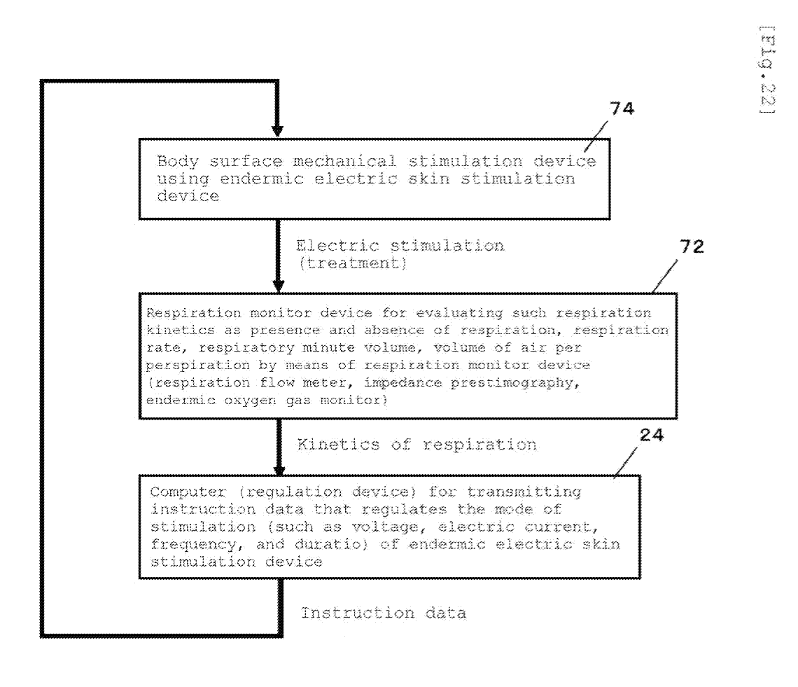

### EXPLANATION OF LETTERS OR NUMERALS

10, 20, 30, 40, 50, 60, 70 . . . . Apparatus for body surface stimulation treatment
12 . . . Vital reaction quantitative evaluation unit
14 . . . Body surface stimulation regulation unit
16 . . . Body surface stimulation unit
22 . . . Electrocardiographic meter
24 . . . Regulating device
26 . . . Electric acupuncture device
28 . . . Patient
32 . . . Blood pressure meter
42 . . . Air conditioner device
52 . . . Massage device
72 . . . Respiration monitor device
74 . . . Endermic electric skin stimulation device

1. An apparatus for body surface stimulation treatment comprising:
a body surface stimulation unit for physically or chemically stimulating the body surface tissue of a patient,
a vital reaction quantitative evaluation unit for quantitatively evaluating the vital reaction of said patient, and
a body surface stimulation regulation unit for regulating stimulation properties to be imparted to the body surface tissue of said patient by said body surface stimulation unit based on a vital data acquired by said vital reaction quantitative evaluation unit.

2. The apparatus for body surface stimulation treatment according to claim 1, wherein
said vital reaction is the autonomic nerve activity, a change of visceral functions caused by the autonomic nerve activity, a vital signal reflecting the change of the visceral functions, or the respiration of said patient.

3. The apparatus for body surface stimulation treatment according to claim 2, wherein
said vital reaction quantitative evaluation unit is a neuroelectric meter capable of measuring said autonomic nerve activity, an electrocardiographic meter capable of measuring at least heart rate and electrocardiographic wave form as said vital signal, a local continuous blood flow meter capable of measuring at least pulse wave form as said vital signal, or a blood pressure meter capable of measuring at least blood pressure and pulse rate as said vital signal.

4. The apparatus for body surface stimulation treatment according to claim 1, wherein said body surface stimulation unit is an electric stimulation means for electrically stimulating the body surface tissue of said patient.

5. The apparatus for body surface stimulation treatment according to claim 4, wherein said electric stimulation means is so configured as to enable regulation of at least one factor selected from the group consisting of voltage, electric current, frequency, frequency of stimulation, and duration of stimulation.

6. The apparatus for body surface stimulation treatment according to claim 1, wherein said body surface stimulation unit is a thermal stimulation unit for thermally stimulating the body surface tissue of said patient.

7. The apparatus for body surface stimulation treatment according to claim 1, wherein said body surface stimulation unit is a mechanical stimulation means for mechanically stimulating the body surface tissue of said patient.

8. The apparatus for body surface stimulation treatment according to claim 7, wherein said mechanical stimulation means is so configured as to enable regulation of at least one factor selected from the group consisting of position of stimulation, intensity of stimulation, frequency of stimulation, and duration of stimulation.

9. The apparatus for body surface stimulation treatment according to claim 1 wherein said body surface stimulation regulation unit is so configured as to enable communication with said vital reaction quantitative evaluation unit or said body surface stimulation unit by means of wireless communication.

10. The apparatus for body surface stimulation treatment according to claim 1, wherein said body surface stimulation regulation unit possesses
a function of outputting a stimulation condition datum and at least one stimulation condition candidate value formed by performing a specific calculation on said stimulation condition datum to said body surface stimulation unit,
a function of retrieving the optimum vital data most suitable for the purpose of therapy among the vital data acquired from said vital reaction quantitative evaluation unit in conformity with the output of said stimulation condition datum or said stimulation condition candidate value, and
a function of setting said stimulation condition datum or said stimulation condition candidate value relating to said optimum vital data as a new stimulation condition datum.

11. The apparatus for body surface stimulation treatment according to claim 1, wherein said body surface stimulation regulation unit possesses a function of continuing the stimulation to the body surface tissue of the patient by said body surface stimulation unit when the vital data acquired by said vital reaction quantitative evaluation unit fits the purpose of therapy and stopping the stimulation to the body surface tissue of the patient by said body surface stimulation unit when the vital data acquired by said vital reaction quantitative evaluation unit does not fit the purpose of therapy.

12. A non-transitory computer readable recording medium on which a program for body surface stimulation treatment in recorded, wherein said program is characterized by causing an apparatus for body surface stimulation treatment to execute
a process (1) for physically or chemically stimulating the body surface tissue of a patient with a body surface stimulation unit,
a process (2) for quantitatively evaluating the vital reaction of said patient with a vital reaction quantitatively evaluating means, and
a process (3) for regulating the stimulation properties to be imparted to the body surface tissue of said patient with said body surface stimulation unit based on the vital data acquired by the process (2).

13. The non-transitory computer readable recording medium according to claim 12, wherein said process (3) comprises
a step of outputting a stimulation condition datum and at least one stimulation condition candidate value formed by performing a specific calculation on said stimulation condition datum to said body surface stimulation unit,
a step of retrieving the optimum vital data most suitable for the purpose of therapy among the vital data acquired from said vital reaction quantitative evaluation unit in conformity with the output of said stimulation condition datum or said stimulation condition datum, and
a step of setting said stimulation condition datum or said stimulation condition candidate value relating to said optimum vital data as a new stimulation datum.

14. The non-transitory computer readable recording medium according to claim 12, wherein said means (3) comprises a step of continuing the stimulation to the body surface tissue of the patient by said body surface stimulation unit when the vital data acquired by said vital reaction quantitative evaluation unit fits the purpose of therapy and stopping the stimulation to the body surface tissue of the patient by said body surface stimulation unit when the vital data acquired by said vital reaction quantitative evaluation unit does not fit the purpose of therapy.

15. (canceled)
